(19) United States
(12) Patent Application Publication
(10) Pub. No.: US 2023/0022147 A1
(43) Pub. Date: Jan. 26, 2023

(54) Optic Nerve Head Oxygen Perfusion as a Real Time Biomarker for Traumatic Brain Injury

(71) Applicant: Joseph Milton Myers, Ann Arbor, MI (US)

(72) Inventor: Joseph Milton Myers, Ann Arbor, MI (US)

(21) Appl. No.: 17/864,791

(22) Filed: Jul. 14, 2022

Related U.S. Application Data
(60) Provisional application No. 63/223,758, filed on Jul. 20, 2021.

(51) Int. Cl. A61B 5/00; A61B 5/1455; G16H 10/20

(52) U.S. Cl. CPC A61B 5/4064; A61B 5/14555; A61B 5/4842; G16H 10/20; A61B 2503/10

(57) ABSTRACT

Methods are provided for measuring real-time concussive and vascular brain injuries, within seconds of the incident, by using a cascading decrease of optic nerve oxygen perfusion as captured by the retina oximetry, which is representative of inherent processes taking place within the brain subsequent to various degrees of cranial insult. The methods presented herein provide for establishing baseline databases and protocols that are intended to set standards for testing and protecting athletes on the field of play and creating emergency guidelines for early treatment of cerebrovascular accidents. Further, methods of investigation presented herein are capable of creating a database of demographically representative values, that are intended to provide statistical norms significant to demographics, such as age, sex, race, location, and athletic endeavors both at rest and within an active athletic state.

This application claims the benefit of and priority to U.S. Provisional Application, entitled “Optic Nerve Head Oxygen Perfusion As A Real Time Biomarker For Traumatic Brain Injury,” filed on Jul. 20, 2021, and having application Ser. No. 63/223,758, the entirety of said application being incorporated herein by reference.

### FIELD

Embodiments of the present disclosure generally relates to oximetry and eye-related measurements. More specifically, embodiments of the disclosure relate to methods for using oximetry to identify traumatic brain injuries.

### BACKGROUND

The U.S. Center for Disease Control (CDC) estimates there are more than 2.5 million emergency room visits in the United States as a result of head injuries, and a study by the CDC and Children's Hospital of Philadelphia showed that 82% of children with concussions report to their primary care doctor rather than the emergency room, suggesting the actual number of concussions occurring in the United States each year could potentially be as high as 10 million. Traumatic brain injury is the leading cause of disability and the number one cause of death for young adults. Automobile accidents, falls, sports-related injuries, and assaults are common causes of traumatic brain injuries. At least 75% of brain injuries in the United States are considered mild traumatic brain injuries, representing an economic burden of at least $16.7 billion annually in direct and indirect costs on the healthcare system. As such, there is a continuous need for methods for using oximetry devices to quickly provide accurate reproducible scans of optic nerve perfusion to determine instances and severity of traumatic brain injuries.

### SUMMARY

Methods are provided for measuring real-time concussive and vascular brain injuries (within seconds of the incident) by using a cascading decrease of optic nerve oxygen perfusion, as measured by the retinal oximetry hypoxia which is representative of inherent processes taking place within the brain subsequent to various degrees of cranial insult. The methods presented herein provide for establishing baseline databases and protocols that are intended to set standards for testing and protecting athletes on the field of play and creating emergency guidelines for early treatment of cerebrovascular accidents. Further, methods of investigation presented herein are capable of creating a database of demographically representative values, that are intended to provide statistical norms significant to demographics, such as age, sex, race, location, and athletic endeavors both at rest and within an active athletic state.

In an exemplary embodiment, a method for correlating a change in oxygenation of an optic nerve/retina to the severity of a traumatic brain injury comprises: obtaining a multiplicity of oximetry devices; surveying a group of test subjects; performing scans of optic nerve/retina oximetry of each test subject; demonstrating that a change in oxygenation of the retinal vasculature coming off the Optic Nerve has a direct correlation to severity of a traumatic brain injury; showing that oxygenation levels of the retinal vasculature can be measured in real-time; demonstrating that the level of hypoxia of the optic nerve or retina indicates the severity of traumatic brain injuries; and using oximetry measurements to be prognostic of rehabilitation periods, goals, and success timelines.

In another exemplary embodiment, performing scans includes using the multiplicity of oximetry devices to perform scan sessions on each test subjects at primary, 2 weeks, 1 month and 3-month sessions. In another exemplary embodiment, performing scans includes using the multiplicity of oximetry devices to scan retinal oximetry during time periods ranging between 1 second and 11 seconds while the test subject is at rest. In another exemplary embodiment, the time periods may be substantially 1, 3, 5, 8, and 10 seconds in duration. In another exemplary embodiment, performing scans includes using the multiplicity of oximetry devices to scan retinal oximetry immediately after physical activity. In another exemplary embodiment, performing scans includes using the multiplicity of oximetry devices to scan retinal oximetry after a time period following physical activity.

In another exemplary embodiment, surveying includes performing a survey of each test subject's demographics, such as, by way of non-limiting example, gender, age, race, sport, competitive season versus off season, and the like. In another exemplary embodiment, surveying includes recruiting athletes as a control group comprising a portion of the group of test subjects. In another exemplary embodiment, performing scans includes performing scans on up to 150 athletes per week. In another exemplary embodiment, performing scans includes performing scans during each athlete's pre-season conditioning and through the competitive season as a small increment of the athlete's fitness training.

In another exemplary embodiment, showing that oxygenation levels of the optic nerve or retina vasculature can be measured in real-time includes showing that the oximetry device accurately captures oxygen perfusion of the optic nerve as measured by the retinal vasculature. In another exemplary embodiment, showing that the oximetry device accurately captures oxygen perfusion of the optic nerve, through measurement of the retinal oximetry includes showing that the oximetry device captures scans that are reproducible within seconds, minutes, hours, days, weeks and months with exception to age variances as an individual progresses in years. In another exemplary embodiment, showing that the oximetry device accurately captures oxygen perfusion of the optic nerve, through measurement of the retinal oximetry includes showing that the oximetry device has an optimal scan time-period that falls between 1 second and about 11 seconds.

In another exemplary embodiment, showing that oxygenation levels of the optic nerve as captured by the retina oximetry can be measured in real-time includes showing that there are demographically standardized norms for gender, age, race, ethnicity, and geographical location. In another exemplary embodiment, showing that oxygenation levels of the optic nerve or retina can be measured in real-time includes showing that oxygen perfusion of the optic nerve, through measurement of the retinal oximetry differs before and after athletic activity. In another exemplary embodiment, showing that oxygen perfusion of the optic nerve, through measurement of the retinal oximetry can be measured in real-time includes showing that concussed scan values show a markedly reduced deviation from an individual's values and a marked deviation from the individual's group norms.

In another exemplary embodiment, performing scans includes measuring the retinal oxygen levels of healthy non-concussed individuals, with differentiation for any ocular or systemic disease states. In another exemplary embodiment, demonstrating includes using resulting baseline values of the non-concussed individuals to investigate active changes during any possible traumatic brain injuries incidents. In another exemplary embodiment, demonstrating includes using the oximetry device to establish concrete quantitative values that are indicative of an individual's concussive cascade during and after a traumatic brain injury. In another exemplary embodiment, demonstrating includes demonstrating that ocular oxygenation depressions can be established and correlated as an indicator of the occurrence and depth of a traumatic brain injury.

These and other features of the concepts provided herein may be better understood with reference to the drawings, description, and appended claims.

### BRIEF DESCRIPTION OF THE DRAWINGS

The drawings refer to embodiments of the present disclosure in which:

FIG. 1 illustrates an exemplary embodiment of an oximetry device that may be used to perform scans of optic nerve perfusion within desired time periods;

FIG. 2 illustrates a flow-chart of objectives of the methods presented herein;

FIG. 3 illustrates a flow-chart of research questions that may be answered by way of the methods presented herein; and

FIG. 4 illustrates a process whereby measurements of optic nerve oximetry may be performed on heathy non-concussed athletes.

While the present disclosure is subject to various modifications and alternative forms, specific embodiments thereof have been shown by way of example in the drawings and will herein be described in detail. The present disclosure should be understood to not be limited to the particular forms disclosed, but on the contrary, the intention is to cover all modifications, equivalents, and alternatives falling within the spirit and scope of the present disclosure.

### DETAILED DESCRIPTION

In the following description, numerous specific details are set forth in order to provide a thorough understanding of the present disclosure. It will be apparent, however, to one of ordinary skill in the art that the methods disclosed herein may be practiced without these specific details. In other instances, specific numeric references such as “first device,” may be made. However, the specific numeric reference should not be interpreted as a literal sequential order but rather interpreted that the “first device” is different than a “second device.” Thus, the specific details set forth are merely exemplary. The specific details may be varied from and still be contemplated to be within the spirit and scope of the present disclosure. The term “coupled” is defined as meaning connected either directly to the component or indirectly to the component through another component. Further, as used herein, the terms “about,” “approximately,” or “substantially” for any numerical values or ranges indicate a suitable dimensional tolerance that allows the part or collection of components to function for its intended purpose as described herein.

In general, methods are provided for measuring real-time concussive and vascular brain injuries (within seconds of the incident) by using a cascading decrease of oxygen perfusion of the optic nerve, through measurement of the retinal oximetry, which is representative of inherent processes taking place within the brain subsequent to various degrees of cranial insult. The methods presented herein provide for establishing baseline databases and protocols that are intended to set standards for testing and protecting athletes on the field of play and creating emergency guidelines for early treatment of cerebrovascular accidents. Further, methods of investigation presented herein are capable of creating a database of demographically representative values, that are intended to provide statistical norms significant to demographics, such as age, sex, race, location, and athletic endeavors both at rest and within an active athletic state.

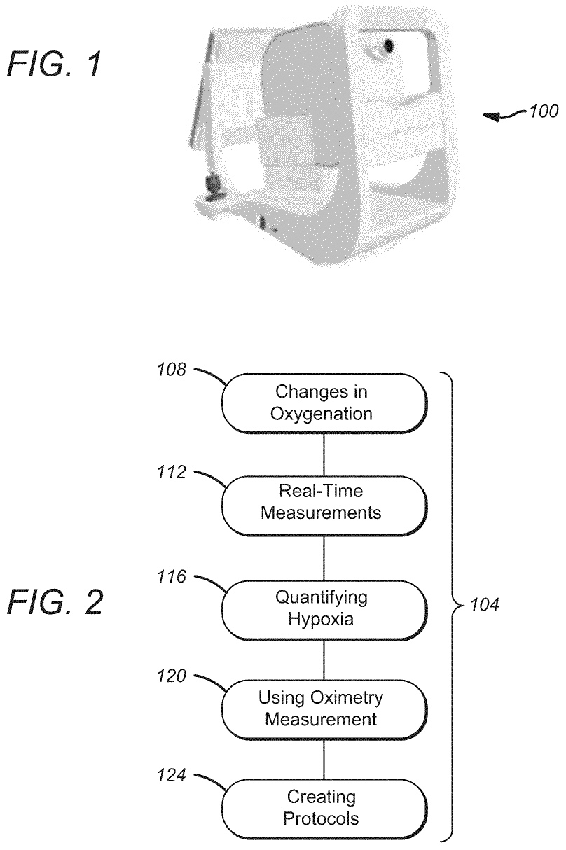

There are no present quantitative field devices that within seconds can provide immediate diagnostic values measuring the effects of a concussive injury. However, an oximetry device, such as the oximetry device 100 shown in FIG. 1, may be used to provide accurate reproducible scans of optic nerve perfusion within a time period ranging between 1 second and 11 seconds and over time frames that reproducibility will prove consequential to the original data collection. Targeting the optic nerve head is difficult, but the oximetry device 100 has been shown to accurately scan with very good reliability and reproducibility. The accuracy of the device 100 enables a standardization of the norms for a diverse demography. It is contemplated that this is essential to the effectiveness of an omnipresent device, process and protocol that serves everyone. It is further contemplated that the device 100 will enable a strong protection of Athletes as an objective determinant of fitness to continue.

FIG. 2 illustrates a flow-chart 104 of objectives of the methods presented herein. In some embodiments, a step 108 comprises demonstrating that a change in oxygenation of the optic nerve retina vasculature has a direct correlation to the severity of a traumatic brain injury. A step 112 comprises, in some embodiments, showing that oxygenation levels of the optic nerve retina vasculature can be measured in real time using the device 100 shown in FIG. 1. Step 116 comprises, in some embodiments, showing that the severity of traumatic brain injuries can be indicated by quantifying the level of hypoxia immediately upon a traumatic brain injury as well as subsequent to initial traumatic brain injuries. Step 120 comprises, in some embodiments, using the oximetry measurements to be prognostic of rehabilitation periods, as well as using oximetry to measure rehabilitative goals and success timelines. In some embodiments, step 124 includes creating protocols for engagement and use for military, athletics and in the Emergency Room.

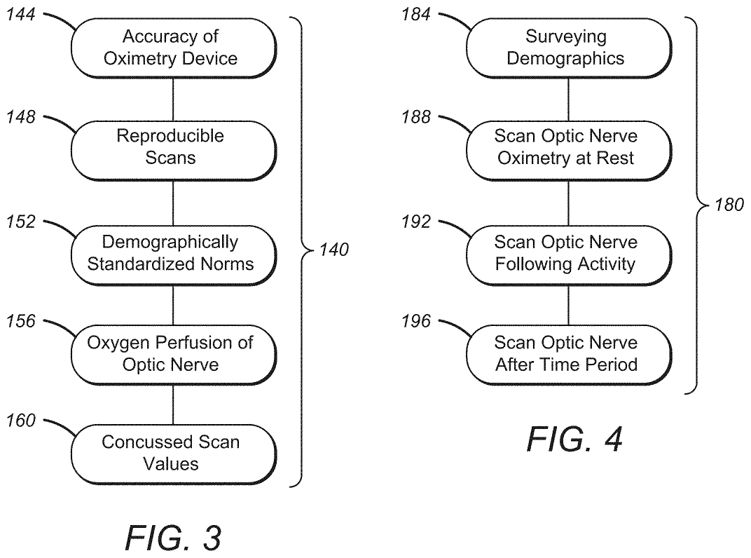

FIG. 3 illustrates a flow-chart 140 of research questions that may, in some embodiments, be answered by way of the methods presented herein. In some embodiments, the methods herein include a step 144 wherein it may be shown that the device 100 accurately captures oxygen levels of the optic nerve. Further, the methods may, in some embodiments, show, in a step 148, that the device 100 captures scans that are reproducible within seconds, minutes, hours, days, weeks and months with exception to age variances as an individual progresses in years. The step 148 may include, in some embodiments, that the device 100 has an optimal scan time-period that falls between 1 second and about 11 seconds. In step 152, the methods may show, in some embodiments, that there are demographically standardized norms for gender, age, race, ethnicity, and geographical location. At a step 156, it may be shown that oxygen perfusion of the optic nerve differs before and after athletic activity. Further, at step 160, it may be shown that concussed scan values show a markedly reduced deviation from an individual's values and a marked deviation from the individual's group norms.

In the methods presented herein, the oximetry device 100 may be used to establish concrete quantitative values, which may be indicative of an individual's concussive cascade during and after a traumatic brain injury. In measuring ocular oxygenation (e.g., the optic nerve head), the ocular oxygenation depressions can be established and correlated as an indicator of the occurrence and depth of the traumatic brain injury. Further, optic nerve oxygen perfusion as captured by the retina oximetry may be used a biomarker to which the breadth and depth of traumatic brain injury is diagnosed. It is contemplated that evaluating the oxygen perfusion as captured by the retina oximetry cascade will become a standard method for providing real-time concussion determination in a plethora of arenas, such as athletics, school safety, vascular insult, geriatric care, pediatric care, military operations, and the like, without limitation.

Presently, conventional methods of evaluating concussive stages includes performing a series of cognitive, observational, and reflective components to establish an intensity and depth of a traumatic brain injury, progressing from diagnosis, through treatment to rehabilitation. It is contemplated, however, that by using the oximetry device 100 in the process of evaluating and rehabilitating traumatic brain injuries, absolute differential values in the concussive stages can be established. Further, reflective recovery values during rehab and statistical norms as baseline objectives for age, gender, race, and ethnicity can be established.

In an embodiment, the method includes measuring the retinal oxygen levels of healthy non-concussed individuals, with differentiation for any ocular or systemic disease states. The traumatic brain injuries investigation may then use resulting baseline values of these individuals to investigate active changes during any possible traumatic brain injuries incidents. It is contemplated that the method may, in some embodiments, use athletic teams as a control group. It is further contemplated that sports teams such as football, hockey and soccer may be particularly useful groups due to a relatively high incidence of concussions reported in such sports.

FIG. 4 illustrates a process 180 whereby measurements of optic nerve oximetry as captured by the retina vasculature may be performed on heathy non-concussed athletes. In the process 180, a multiplicity of devices 100 may be used to perform scan sessions on each athlete at primary, 2 weeks, 1 month and 3-month sessions. At step 184, a survey may be performed of each athlete's demographics, such as, by way of non-limiting example, gender, age, race, sport, competitive season versus off season, and the like. In some embodiments, step 188 may comprise using the devices 100 to scan optic nerve oximetry during time periods ranging between 1 second and 11 seconds while the athlete are at rest. In some embodiments, the time periods may be substantially 1, 3, 5, 8, and 10 seconds in duration.

In step 192 of the process 180, the devices may be used to perform scans of optic nerve oximetry after physical activity, such as following a 100-meter dash. The scans may be performed during time periods ranging between 1 second and 11 second. As with step 188, in step 192 the time periods may be substantially 1, 3, 5, 8, and 10 seconds in duration. In step 196, the scan may be performed at a time period following the physical activity. In one exemplary embodiment, the scans are performed 10 minutes after a 100-meter dash for time periods of substantially 1, 3, 5, 8, and 10 seconds in duration. It is contemplated that in some embodiments, three devices 100 may be set up on either side of the 100-meter track markings, and scans may be performed on up to 150 athletes per week.

Moreover, in some embodiments, the process 180 may be performed during an athlete's pre-season conditioning and extend through the competitive season as a small increment of the athlete's fitness training. The process 180 preferably is configured to have the least intrusion into the athlete's life and athletic endeavors. It is contemplated that, outside of providing external and internal anonymity, there should not be any ethical issues in the first round of testing.

It is envisioned that significant outcomes of the methods presented herein demonstrates that the oxygen cascade during and after a traumatic brain injury has a measurable and quantitative value. This value may be used in the diagnosis of a concussion (and its severity), thus providing valuable information in the prognosis of rehabilitation and the time frame as to when the subject may or may not return to their functional duties.

It is contemplated that the methods presented herein will strengthen the model evaluating the decrease in concussed cranial oxygen perfusion by using measurement of the optic nerve (a part of the brain) through a non-invasive, real-time scanning device, such as the device 100, that can be implemented as a portable test in the field of play. Further, the methods herein may encourage investigation into correlations between normal optic nerve perfusion as captured by the retina oximetry and stroke diagnosis (with recombinant tissue plasminogen activator (tPA) treatment). Further, the methods herein may serve as a baseline to investigate optic nerve perfusion as captured by the retina oximetry and a correlation to systemic childhood and geriatric diseases and illnesses.

While the methods have been described in terms of particular variations and illustrative figures, those of ordinary skill in the art will recognize that the methods are not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process, when possible, as well as performed sequentially as described above. To the extent there are variations of the invention, which are within the spirit of the disclosure or equivalent to the invention found in the claims, it is the intent that this patent will cover those variations as well. Therefore, the present disclosure is to be understood as not limited by the specific embodiments described herein, but only by scope of the appended claims.

What is claimed is:

1. A method for correlating a change in oxygenation of an optic nerve/retina to the severity of a traumatic brain injury, comprising:
obtaining a multiplicity of oximetry devices;
surveying a group of test subjects;
performing scans of optic nerve/retinal oximetry of each test subject;
demonstrating that a change in oxygenation of the optic nerve/retina has a direct correlation to severity of a traumatic brain injury;
showing that oxygenation levels of the optic nerve or retina can be measured in real-time;
demonstrating that the level of hypoxia of the optic nerve/retina indicates the severity of traumatic brain injuries; and
using oximetry measurements to be prognostic of rehabilitation periods, goals, and success timelines.

2. The method of claim 1, wherein performing scans includes using the multiplicity of oximetry devices to perform scan sessions on each test subjects at primary, 2 weeks, 1 month and 3-month sessions.

3. The method of claim 2, wherein performing scans includes using the multiplicity of oximetry devices to scan optic nerve oximetry during time periods ranging between 1 second and 11 seconds while the test subject is at rest.

4. The method of claim 3, wherein the time periods may be substantially 1, 3, 5, 8, and 10 seconds in duration.

5. The method of claim 4, wherein performing scans includes using the multiplicity of oximetry devices to scan optic nerve/retinal oximetry immediately after physical activity.

6. The method of claim 5, wherein performing scans includes using the multiplicity of oximetry devices to scan optic nerve/retinal oximetry after a time period following physical activity.

7. The method of claim 1, wherein surveying includes performing a survey of each test subject's demographics, such as, by way of non-limiting example, gender, age, race, sport, competitive season versus off season, and the like.

8. The method of claim 1, wherein surveying includes recruiting athletes as a control group comprising a portion of the group of test subjects.

9. The method of claim 8, wherein performing scans includes performing scans on up to 150 athletes per week.

10. The method of claim 9, wherein performing scans includes performing scans during each athlete's pre-season conditioning and through the competitive season as a small increment of the athlete's fitness training.

11. The method of claim 1, wherein showing that oxygenation levels of the optic nerve/retina can be measured in real-time includes showing that the oximetry device accurately captures oxygen levels of the optic nerve.

12. The method of claim 11, wherein showing that the oximetry device accurately captures oxygen levels of the optic nerve/retina includes showing that the oximetry device captures scans that are reproducible within seconds, minutes, hours, days, weeks and months with exception to age variances as an individual progresses in years.

13. The method of claim 12, wherein showing that the oximetry device accurately captures oxygen levels of the optic nerve/retina includes showing that the oximetry device has an optimal scan time-period that falls between 1 second and about 11 seconds.

14. The method of claim 12, wherein showing that oxygenation levels of the optic nerve or retina can be measured in real-time includes showing that there are demographically standardized norms for gender, age, race, ethnicity, and geographical location.

15. The method of claim 12, wherein showing that oxygenation levels of the optic nerve/retina can be measured in real-time includes showing that oxygen perfusion of the optic nerve as captured by the retina oximetry differs before and after athletic activity.

16. The method of claim 12, wherein showing that oxygenation levels of the optic nerve/retina can be measured in real-time includes showing that concussed scan values show a markedly reduced deviation from an individual's values and a marked deviation from the individual's group norms.

17. The method of claim 1, wherein performing scans includes measuring the retinal oxygen levels of healthy non-concussed individuals, with differentiation for any ocular or systemic disease states.

18. The method of claim 17, wherein demonstrating includes using resulting baseline values of the non-concussed individuals to investigate active changes during any possible traumatic brain injuries incidents.

19. The method of claim 1, wherein demonstrating includes using the oximetry device to establish concrete quantitative values that are indicative of an individual's concussive cascade during and after a traumatic brain injury.

20. The method of claim 1, wherein demonstrating includes demonstrating that ocular oxygenation depressions can be established and correlated as an indicator of the occurrence and depth of a traumatic brain injury.
